(19) United States
(12) Patent Application Publication
(10) Pub. No.: US 2018/0360640 A1
(43) Pub. Date: Dec. 20, 2018

(54) Support Garment Assembly

(71) Applicants: Bernadette Brown, Port St. Lucie, FL (US); Lawrence E. Brown, Port St. Lucie, FL (US)

(72) Inventors: Bernadette Brown, Port St. Lucie, FL (US); Lawrence E. Brown, Port St. Lucie, FL (US)

(21) Appl. No.: 15/624,852

(22) Filed: Jun. 16, 2017

(51) Int. Cl. A61F 5/02

(52) U.S. Cl. CPC A61F 5/028

(57) ABSTRACT

A support garment assembly for supporting a lower back includes a belt that may be worn around a waist thereby facilitating the belt to extend along a lower back. The belt is comprised of a resiliently stretchable material to support the lower back thereby treating symptoms of a lower back injury. A strap is removably coupled to the belt and the strap extends beneath a crotch. In this way the strap inhibits the belt from riding upwardly on the waist.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to garment devices and more particularly pertains to a new garment device for supporting a lower back.

### BRIEF SUMMARY OF THE INVENTION

An embodiment of the disclosure meets the needs presented above by generally comprising a belt that may be worn around a waist thereby facilitating the belt to extend along a lower back. The belt is comprised of a resiliently stretchable material to support the lower back thereby treating symptoms of a lower back injury. A strap is removably coupled to the belt and the strap extends beneath a crotch. In this way the strap inhibits the belt from riding upwardly on the waist.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a support garment assembly according to an embodiment of the disclosure.

FIG. 2 is a back exploded view of an embodiment of the disclosure.

FIG. 3 is a bottom view of a belt of an embodiment of the disclosure.

FIG. 4 is a front view of an alternative embodiment of the disclosure.

FIG. 5 is a perspective in-use view of an embodiment of the disclosure.

FIG. 6 is a perspective view of an alternative embodiment of the disclosure.

FIG. 7 is a right side view of a strap of an embodiment of the disclosure.

### DETAILED DESCRIPTION OF THE INVENTION

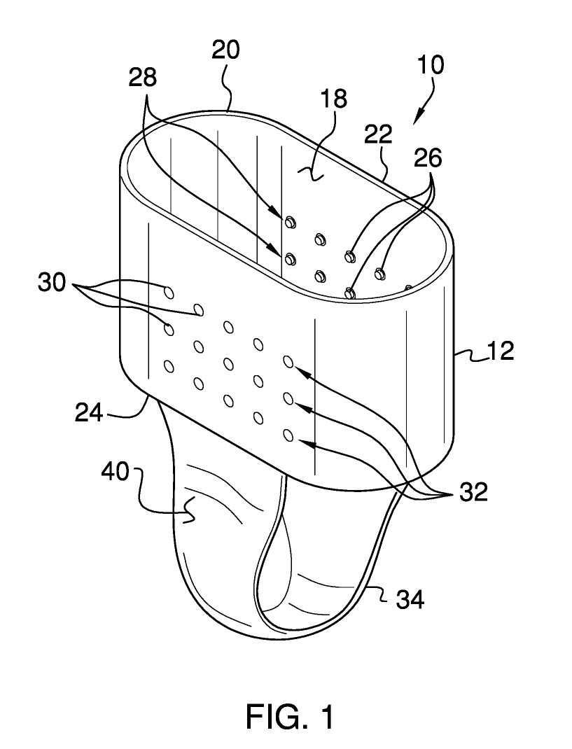

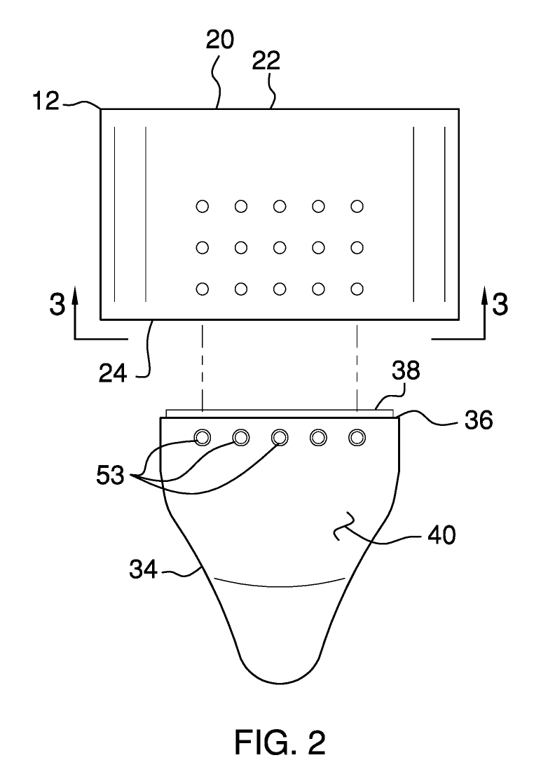

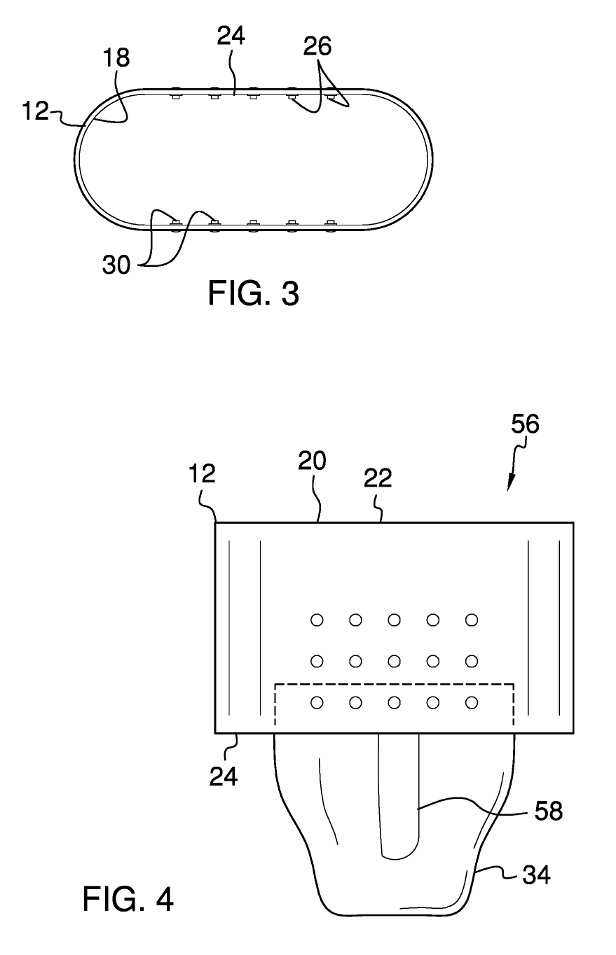

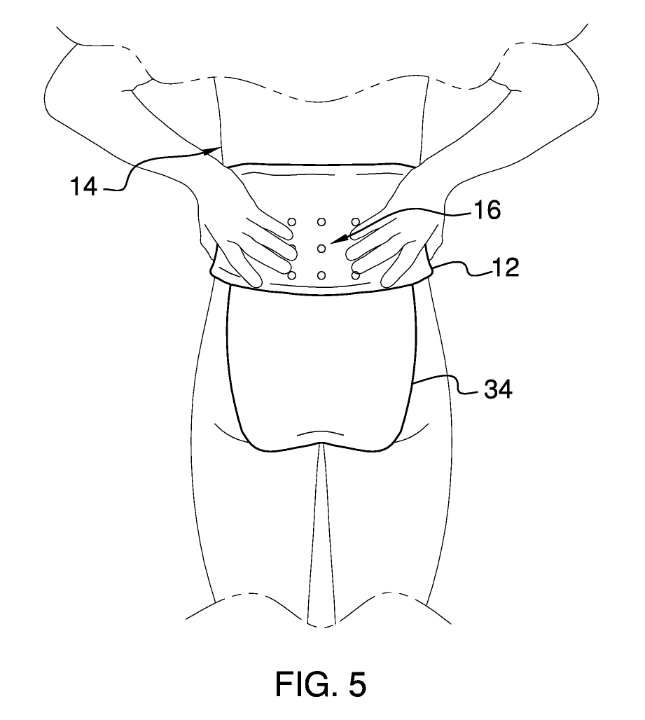

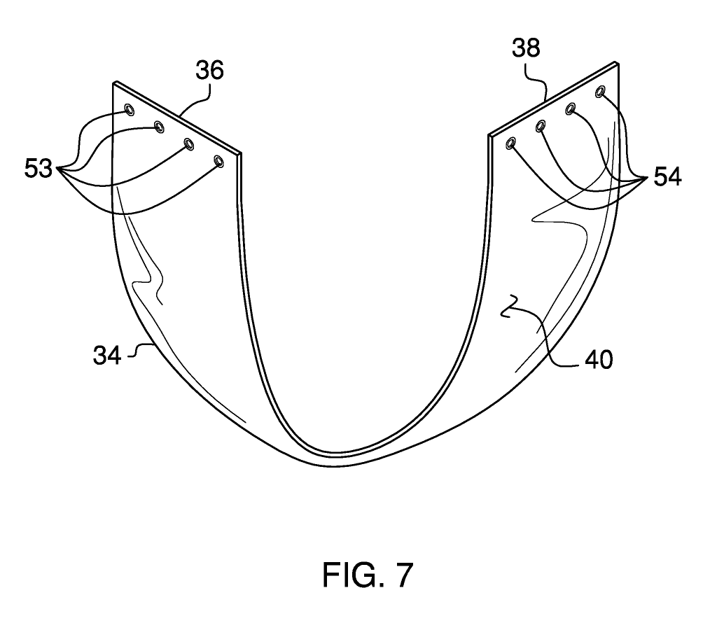

With reference now to the drawings, and in particular to FIGS. 1 through 7 thereof, a new garment device embodying the principles and concepts of an embodiment of the disclosure and generally designated by the reference numeral 10 will be described.

As best illustrated in FIGS. 1 through 7, the support garment assembly 10 generally comprises a belt 12 that may be worn around a waist 14 thereby facilitating the belt 12 to extend along a lower back 16. The belt 12 is comprised of a resiliently stretchable material such as elastic or the like. Thus, the belt 12 supports the lower back 16 thereby treating symptoms of a lower back injury. The belt 12 has a first surface 18 and a peripheral edge 20 and the peripheral edge 20 has a top side 22 and a bottom side 24. The first surface 18 abuts the waist 14 when the belt 12 is worn.

A plurality of first fasteners 26 is provided and each of the first fasteners 26 is coupled to the first surface 18. The first fasteners 26 are spaced apart from each other and are arranged into a plurality of rows 28. Additionally, the rows 28 are distributed between the top side 22 and the bottom side 24 of the belt 12. Each of the first fasteners 26 may comprise snaps and any other fastener.

A plurality of second fasteners 30 is provided and each of the second fasteners 30 is coupled to the first surface 18. The second fasteners 30 are spaced apart from each other and are arranged into a plurality of rows 32. The rows 32 corresponding to the second fasteners 30 are distributed between the top side 22 and the bottom side 24 of the belt 12. Additionally, the plurality of second fasteners 30 is positioned on an opposite side of the belt 12 with respect to the first fasteners 26. Each of the second fasteners 30 may comprise a snap and any other fastener.

A strap 34 is provided and the strap 34 is removably coupled to the belt 12. The strap 34 extends beneath a crotch 35 thereby inhibiting the belt 12 from riding upwardly on the waist 14. The strap 34 has a first end 36, a second end 38 and a primary surface 40 extending therebetween. A plurality of third fasteners 53 is provided and each of the third fasteners 53 is coupled to the primary surface 40 of the strap 34. The third fasteners 53 are spaced apart from each other and are distributed along the first end 36 of the strap 34. The strap 34 may narrow between each of the first 36 and second 38 ends to enhance comfort when the strap 34 is worn on the crotch 35.

Each of the third fasteners 53 releasably engages selected ones of the first fasteners 26. Additionally, each of the third fasteners 53 engages a selected one of the rows 28 of the first fasteners 26. Thus, the first end 36 of the strap 34 is spaced a selected distance from the top side 22 of the belt 12. Each of the third fasteners 53 may comprise a snap and any other fastener.

A plurality of fourth fasteners 54 is provided and each of the fourth fasteners 54 is coupled to the primary surface 40 of the strap 34. The fourth fasteners 54 are spaced apart from each other and are distributed along the second end 38 of the strap 34. Each of the fourth fasteners 54 releasably engages selected ones of the second fasteners 30. Additionally, each of the fourth fasteners 54 engages a selected one of the rows 32 of the second fasteners 30. Thus, the second end 38 of the strap 34 is spaced a selected distance from the top side 22 of the belt 12. In this way the strap 34 accommodates a variety of sizes of users.

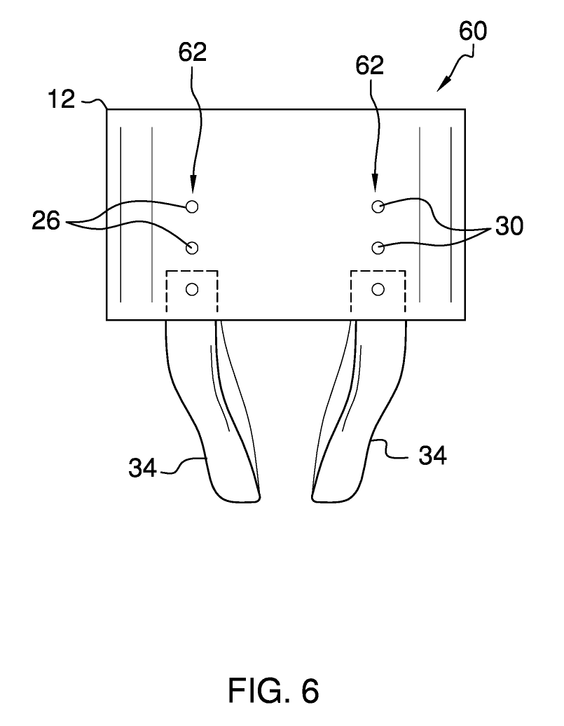

In an alternative embodiment 56 as shown in FIG. 4, the strap 34 may have a fly 58 extending therethrough. Thus, a male user may urinate while the strap 34 and the belt 12 are being worn. In an alternative embodiment 60 as shown in FIG. 6, each of the first 26 and second 30 fasteners may be arranged in a pair of columns 62. A pair of the straps 34 may be provided and each of the straps 34 may be worn around legs of the user. Moreover, the pair of straps 34 engages selected ones of the first 26 and second 30 fasteners.

In use, the belt 12 is worn around the waist 14 and the strap 34 is positioned to extend along the crotch 35. Each of the third fasteners 53 is manipulated to engage a selected row of the first fasteners 26. Each of the fourth fasteners 54 is manipulated to engage a selected row of the second fasteners 30. Thus, the strap 34 inhibits the belt 12 from riding upwardly on the waist 14 thereby facilitating the belt 12 to continuously support the lower back 16. Additionally, the third and fourth fasteners 54 engage the selected row of first and second fasteners 30 to accommodate a variety of sizes of users.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A support garment assembly being configured to be worn around a waist to support a lower back, said assembly comprising:
a belt being configured to be worn around a waist thereby facilitating said belt to extend along a lower back, said belt being comprised of a resiliently stretchable material wherein said belt is configured to support the lower back thereby inhibiting symptoms of a lower back injury; and
a strap being removably coupled to said belt wherein said strap is configured to extending beneath a crotch thereby inhibiting said belt from riding upwardly on the waist.

2. The assembly according to claim 1, further comprising said belt has a first surface and a peripheral edge, said peripheral edge having a top side and a bottom side, said first surface being configured to abut the waist.

3. The assembly according to claim 2, further comprising a plurality of first fasteners, each of said first fasteners being coupled to said first surface, each of said first fasteners being spaced apart from each other and being arranged into a plurality of rows, said rows being distributed between said top side and said bottom side of said belt.

4. The assembly according to claim 2, further comprising:
a plurality of first fasteners; and
a plurality of second fasteners, each of said second fasteners being coupled to said first surface, each of said second fasteners being spaced apart from each other and being arranged into a plurality of rows, said rows corresponding to said second fasteners being distributed between said top side and said bottom side of said belt, said plurality of second fasteners being positioned on an opposite side of said belt with respect to said first fasteners.

5. The assembly according to claim 3, further comprising:
said strap having a first end and a primary surface; and
a plurality of third fasteners, each of said third fasteners being coupled to said primary surface of said strap, said plurality of third fasteners being spaced apart from each other and being distributed along said first end of said strap.

6. The assembly according to claim 5, wherein each of said third fasteners releasably engages selected ones of said first fasteners, each of said third fasteners engaging a selected one of said rows of said first fasteners such that said first end of said strap is spaced a selected distance from said top side of said belt.

7. The assembly according to claim 4, further comprising:
said strap having a second end and a primary surface; and
a plurality of fourth fasteners, each of said fourth fasteners being coupled to said primary surface of said strap, said plurality of fourth fasteners being spaced apart from each other and being distributed along said second end of said strap.

8. The assembly according to claim 7, wherein each of said fourth fasteners releasably engages selected ones of said second fasteners, each of said fourth fasteners engaging a selected one of said rows of said second fasteners such that said second end of said strap is spaced a selected distance from said top side of said belt wherein said strap is configured to accommodate a variety of sizes of users.

9. A support garment assembly being configured to be worn around a waist to support a lower back, said assembly comprising:
a belt being configured to be worn around a waist thereby facilitating said belt to extend along a lower back, said belt being comprised of a resiliently stretchable material wherein said belt is configured to support the lower back thereby inhibiting symptoms of a lower back injury, said belt having a first surface and a peripheral edge, said peripheral edge having a top side and a bottom side, said first surface being configured to abut the waist;
a plurality of first fasteners, each of said first fasteners being coupled to said first surface, each of said first fasteners being spaced apart from each other and being arranged into a plurality of rows, said rows being distributed between said top side and said bottom side of said belt;
a plurality of second fasteners, each of said second fasteners being coupled to said first surface, each of said second fasteners being spaced apart from each other and being arranged into a plurality of rows, said rows corresponding to said second fasteners being distributed between said top side and said bottom side of said belt, said plurality of second fasteners being positioned on an opposite side of said belt with respect to said first fasteners;
a strap being removably coupled to said belt wherein said strap is configured to extending beneath a crotch thereby inhibiting said belt from riding upwardly on the waist, said strap having a first end, a second end and a primary surface extending therebetween;
a plurality of third fasteners, each of said third fasteners being coupled to said primary surface of said strap, said plurality of third fasteners being spaced apart from each other and being distributed along said first end of said strap, each of said third fasteners releasably engaging selected ones of said first fasteners, each of said third fasteners engaging a selected one of said rows of said first fasteners such that said first end of said strap is spaced a selected distance from said top side of said belt; and
a plurality of fourth fasteners, each of said fourth fasteners being coupled to said primary surface of said strap, said plurality of fourth fasteners being spaced apart from each other and being distributed along said second end of said strap, each of said fourth fasteners releasably engaging selected ones of said second fasteners, each of said fourth fasteners engaging a selected one of said rows of said second fasteners such that said second end of said strap is spaced a selected distance from said top side of said belt wherein said strap is configured to accommodate a variety of sizes of users.
